(19) United States
(12) Patent Application Publication
(10) Pub. No.: US 2021/0077288 A1
(43) Pub. Date: Mar. 18, 2021

(54) ASSIST DEVICE

(71) Applicant: Honda Motor Co.,Ltd., Tokyo (JP)

(72) Inventor: Kei SHIMADA, Saitama (JP)

(73) Assignee: Honda Motor Co.,Ltd., Tokyo (JP)

(21) Appl. No.: 17/010,850

(22) Filed: Sep. 3, 2020

(30) Foreign Application Priority Data
Sep. 13, 2019 (JP) 2019-167513

(51) Int. Cl. A61F 5/01

(52) U.S. Cl. CPC A61F 5/0127; A61F 2005/0172; A61F 2005/0158; A61F 2005/0197

(57) ABSTRACT

Provided is an assist device, including a lumbar orthosis, a buttock orthosis, an ankle orthosis, a leg frame and an assist mechanism. In response to a user's bending and stretching motions, the assist mechanism applies an assist force to the user's body by operating the leg frame and separating the lumbar orthosis and the buttock orthosis from the ankle orthosis. The ankle orthosis includes a front locking part and a back locking part configured to be locked to the user's lower leg, as well as an instep abutting part and a heel abutting part abutting against the user's foot from above.

### CROSS-REFERENCE TO RELATED APPLICATIONS

This application claims the priority benefit of Japan Application No. 2019-167513, filed on Sep. 13, 2019. The entirety of the above-mentioned patent application is hereby incorporated by reference herein and made a part of this specification.

### BACKGROUND

### Technical Field

The disclosure relates to an assist device, the assist device applying to a user's body an assist force assisting the user with bending and stretching motions.

### Related Art

Conventionally, various assist devices have been proposed in order to assist the motion of a user such as an elderly person or the like. For example, there is an assist device assisting the user with bending and stretching motions by assisting with the bending and stretching of the user's leg.

As such an assist device assisting with the bending and stretching motions, there has been known one including a lumbar orthosis worn on the user's lumbar region, a foot orthosis worn on the user's foot, a frame connecting the lumbar orthosis and the foot orthosis, and an assist mechanism moving the foot orthosis with respect to the lumbar orthosis (for example, see Patent Document 1).

In this assist device, when assisting the user with the bending and stretching motions, an assist force is applied to the user's body via the lumbar orthosis and the foot orthosis. Here, in the assist device of Patent Document 1, as the foot orthosis, a type which carries the user's foot is adopted. Hence, in this assist device, a force for generating the assist force is also applied to the user's sole.

### PATENT DOCUMENTS

[Patent Document 1] Japanese Patent Laid-open No. 2019-77000

By the way, when the force for generating the assist force is applied to the sole, sometimes the center of pressure of the user's foot may shift due to the above force. Hence, in the assist device of Patent Document 1, there is a fear that, in a state in which the assist force is applied to the user's body, the force applied to the user's sole may make it difficult for the user to maintain balance.

### SUMMARY

An assist device of the disclosure includes: a first orthosis worn on a portion of a body of a user above a knee joint; a second orthosis worn on a lower leg of the user; a leg frame connecting the first orthosis and the second orthosis; and an assist mechanism, in response to bending and stretching motions of a leg of the user, applying an assist force that assists with the bending and stretching motions to the body of the user by operating the leg frame and separating the first orthosis and the second orthosis. The second orthosis includes: a locking part, configured to be locked to the lower leg of the user; and an abutting part, abutting against a foot of the user from above in a state in which the assist force is applied.

### BRIEF DESCRIPTION OF THE DRAWINGS

FIG. 1 is a perspective view showing a configuration of an assist device according to a first embodiment.

FIG. 2 is a back view of the assist device of FIG. 1.

FIG. 3 is a side view of the assist device of FIG. 1, showing a case where a user in a wearing state is standing.

FIG. 4 is a side view of the assist device of FIG. 1, showing a case where the user in the wearing state slightly bends a knee.

FIG. 5 is a side view of the assist device of FIG. 1, showing a case where the user in the wearing state largely bends the knee.

FIG. 6 is a perspective view enlarging and showing a structure of a foot orthosis of the assist device of FIG. 1.

FIG. 7 is a side view from the inside, showing a part of the foot orthosis of the assist device of FIG. 1 in cross section.

FIG. 8 is a plan view of the foot orthosis of the assist device of FIG. 1.

FIG. 9 is a front view of the foot orthosis of the assist device of FIG. 1.

FIG. 10 is a perspective view from the front side, schematically showing a configuration of a leg frame and a foot orthosis of an assist device according to a second embodiment.

FIG. 11 is a side view schematically showing a configuration of the assist device of FIG. 10, showing a case where the user in the wearing state is standing.

FIG. 12 is a side view schematically showing the configuration of the assist device of FIG. 10, showing a case where the user in the wearing state begins to bend a knee.

FIG. 13 is a side view schematically showing the configuration of the assist device of FIG. 10, showing a case where the user in the wearing state has bent the knee.

FIG. 14 is a perspective view from the front side, schematically showing a configuration of a leg frame and a foot orthosis of an assist device according to a third embodiment.

FIG. 15 is a side view schematically showing a configuration of the assist device of FIG. 14, showing a case where the user in the wearing state is standing.

FIG. 16 is a side view schematically showing the configuration of the assist device of FIG. 14, showing a case where the user in the wearing state begins to bend a knee.

FIG. 17 is a side view schematically showing the configuration of the assist device of FIG. 14, showing a case where the user in the wearing state has bent the knee.

FIG. 18 is a perspective view from the front side, schematically showing a configuration of a leg frame and a foot orthosis of an assist device according to a fourth embodiment.

FIG. 19 is a schematic view showing a configuration of a tracking mechanism of the assist device of FIG. 18, showing a case where the user in the wearing state is bending a knee.

FIG. 20 is a schematic view showing the configuration of the tracking mechanism of the assist device of FIG. 18, showing a case where the user in the wearing state is stretching the knee.

FIG. 21 is a side view schematically showing a configuration of the assist device of FIG. 18, showing a case where the user in the wearing state is standing.

FIG. 22 is a side view schematically showing the configuration of the assist device of FIG. 18, showing a case where the user in the wearing state begins to bend the knee.

FIG. 23 is a side view schematically showing the configuration of the assist device of FIG. 18, showing a case where the user in the wearing state has bent the knee.

### DESCRIPTION OF THE EMBODIMENTS

The disclosure provides an assist device in which a user easily maintains balance in a state in which an assist force is applied to the user's body.

The assist device of the disclosure includes: a first orthosis worn on a portion of a body of a user above a knee joint; a second orthosis worn on a lower leg of the user; a leg frame connecting the first orthosis and the second orthosis; and an assist mechanism, in response to bending and stretching motions of a leg of the user, applying an assist force that assists with the bending and stretching motions to the body of the user by operating the leg frame and separating the first orthosis and the second orthosis. The second orthosis includes: a locking part, configured to be locked to the lower leg of the user; and an abutting part, abutting against a foot of the user from above in a state in which the assist force is applied.

In this way, in the assist device of the disclosure, the second orthosis abuts against the user's foot from above via the abutting part in an assist force application state in which the assist device operates so as to apply the assist force to the user's body. Specifically, the second orthosis abuts against, for example, an upper surface of the foot, such as an instep, an upper surface of a portion inclined forward above a heel, or the like.

Accordingly, in the assist force application state, a force for generating the assist force is applied to the portion (for example, lumbar region, abdomen, armpit, the whole upper body or the like) of the user's body above the knee joint that wears the first orthosis and to the upper surface of the foot in abutting contact with the second orthosis. That is, no force is applied to the user's sole.

Therefore, in the assist device of the disclosure, by the force for generating the assist force, the center of pressure (hereinafter “COP”) of the user's foot is prevented from shifting. Thus, the user easily maintains balance even in the assist force application state.

By the way, in the conventional assist device as described in Patent Document 1, the weight of a member constituting the assist device is configured to be released to a ground plane. Specifically, in the conventional assist device, the type that carries the foot is adopted as the foot orthosis, and the weight of a frame or the like connected to the foot orthosis is released to the ground plane via the foot orthosis.

In contrast, in the assist device of the disclosure, the first orthosis is worn on the portion of the user's body above the knee joint, the second orthosis is worn on the user's lower leg, and the leg frame connects the first orthosis and the second orthosis. That is, the weight of the leg frame and the second orthosis can be supported by the upper portion of the user's body via the first orthosis.

Accordingly, in the assist device, there is no need to provide a member for grounding the assist device and release the weight of the member constituting the assist device to the ground plane. Thus, there is no need to adopt the type that carries the foot as the foot orthosis.

Therefore, according to the assist device of the disclosure, since there is no need to adopt the type that carries the foot as the foot orthosis, a structure located below an ankle can be downsized, and the degree of freedom of motion of an ankle joint of the user in a state (hereinafter sometimes simply “wearing state”) in which the assist device is worn on the user can also be improved.

Preferably, in the assist device of the disclosure, a foot frame extending laterally from the leg frame and rotatably connected to the leg frame about a pitch axis is provided. The locking part includes: a front locking part, configured to be locked to a front surface side of the lower leg of the user; and a back locking part, configured to be locked to a back surface side of the lower leg of the user. The second orthosis is connected to the leg frame via the foot frame. The foot frame holds the front locking part on a front side and holds the back locking part on a rear side. A connected portion between the leg frame and the foot frame is located between the front locking part and the back locking part in side view.

With such a configuration, in the wearing state, the connected portion between the leg frame and the foot frame is located close to the user's ankle in a front-rear direction in side view. Accordingly, a difference between the motion of the foot frame (and thus the abutting part that applies force to the user) and the motion of the user's foot is reduced. As a result, a deviation of the abutting part from the user's foot is suppressed, and a shift in the COP in the assist force application state can also be reduced. Thus, the COP is easily kept within a range of a support base surface.

Preferably, in the assist device of the disclosure, a foot frame extending laterally from the leg frame and rotatably connected to the leg frame about a pitch axis is provided. The second orthosis is connected to the leg frame via the foot frame. The locking part is configured to be locked to an ankle of the user. A connected portion between the leg frame and the foot frame is located below the locking part.

With such a configuration, in the wearing state, the connected portion between the leg frame and the foot frame is located close to the user's ankle in an up-down direction. Accordingly, a difference between the motion of the foot frame (and thus the abutting part that applies force to the user) and the motion of the user's foot is reduced. As a result, a deviation of the abutting part from the user's foot is suppressed, and a shift in the COP in the assist force application state can also be reduced. Thus, the COP is easily kept within the range of the support base surface.

Preferably, in the assist device of the disclosure, the locking part is configured to be locked to an ankle of the user. The abutting part extends from a lower end of the locking part.

With such a configuration, since a member for connecting the locking part and the abutting part becomes unnecessary, the size, weight and cost can further be reduced.

Preferably, in the assist device of the disclosure, when the abutting part is configured to extend from the locking part, the locking part is connected to the leg frame.

A force is transmitted from the leg frame to the abutting part of the second orthosis. At this time, the greater the distance from a connected portion between the abutting part and the leg frame to the abutting part, the greater the force applied from above from the abutting part to the user's foot in the assist force application state. Thus, due to the abutting contact with the abutting part, discomfort felt in the foot by the user is also increased.

In order to suppress this discomfort, since the distance from the connected portion to the abutting part may be shortened, the leg frame is preferably directly connected to the abutting part. However, depending on the structure of the assist device, sometimes the leg frame cannot be directly connected to the abutting part.

Therefore, in the case where the abutting part is configured to extend from the locking part, when the locking part is configured to be connected to the leg frame in this way, the distance between the abutting part and the leg frame can be made as small as possible. Thus, by reducing the force applied from above from the abutting part to the user's foot in the assist force application state, the discomfort felt by the user due to the abutting contact with the abutting part can be suppressed.

In the assist device of the disclosure, when the abutting part is configured to extend from the locking part, the locking part may include a front locking part configured to be locked to a front surface side of the lower leg of the user, and the abutting part may be configured to include an instep abutting part abutting against the instep of the user.

In the assist device of the disclosure, when the abutting part is configured to extend from the locking part, the locking part may include a back locking part configured to be locked to a back surface side of the lower leg of the user, and the abutting part may be configured to include a heel abutting part abutting against the foot or the ankle of the user from a heel side.

Preferably, in the assist device of the disclosure, the abutting part is located on a side of the leg frame and is configured to be non-rotatable about a roll axis.

When the abutting part is located on the side of the leg frame, there is a fear that the abutting part may rotate about the roll axis along the curve of the upper surface of the user's foot due to the force for generating the assist force. Therefore, with such a configuration, even if the abutting part is located on the side of the leg frame, the rotation of the abutting part about the roll axis can be suppressed.

Preferably, in the assist device of the disclosure, the abutting part includes a collar extending from the abutting part so as to be located on a side surface of the foot of the user in the wearing state.

When the force for generating the assist force is applied to the foot from above via the abutting part, since the upper surface of the foot is curved, there is a fear that the abutting part may rotate along the curve due to the force. Therefore, when such a collar is provided, the collar abuts against the side surface of the user's foot during the rotation, whereby the rotation of the abutting part can be suppressed.

Preferably, in the assist device of the disclosure, when the abutting part includes the collar, the abutting part includes a heel abutting part abutting against the foot or an ankle of the user from a heel side, and the collar extends forward from the heel abutting part.

In general, when the user moves the foot about the ankle joint, the amount of movement of the rearfoot (portion on the heel side) is smaller than the amount of movement of the forefoot (portion on the instep side). Hence, when the collar is provided on the heel abutting part abutting against the user's foot or ankle from the heel side (that is, abutting against the rearfoot or its vicinity from the back surface side), it is easy to maintain the state in which the foot and the collar abut against each other. Accordingly, the rotation of the abutting part can be stably suppressed.

Preferably, in the assist device of the disclosure, the abutting part includes an instep abutting part abutting against an instep of the user. The instep abutting part has a first curved shape convex upward in front view.

When the force for generating the assist force is applied to the instep via the abutting part, since the instep is curved so as to be convex upward in front view, there is a fear that the instep abutting part may rotate about the roll axis due to the force. Since the instep is inclined toward a toe side, there is also a fear that the instep abutting part may rotate about a yaw axis due to the force.

Therefore, when the shape of the instep abutting part is made to be a curved shape convex upward in front view in this way, the instep abutting part may firmly abut against the instep in the assist force application state. Accordingly, the rotation of the instep abutting part can be suppressed.

Preferably, in the assist device of the disclosure, when the instep abutting part has the first curved shape convex upward in front view, the instep abutting part is located on a side of the leg frame. The instep abutting part includes a first portion located farther from the leg frame than an apex of the first curved shape in front view, and a second portion connected to the first portion and located closer to the leg frame than the apex of the first curved shape in front view. The first portion has a smaller radius of curvature than the second portion.

When the instep abutting part is located on the side of the leg frame, there is a fear that the instep abutting part may be attracted to the leg frame due to the force for generating the assist force. Therefore, when a curvature of the first portion in a position away from the leg frame is reduced in this way, since the first portion easily abuts against a side surface of the user's foot opposite the leg frame, the movement of the instep abutting part can be suppressed at the abutted portion.

Preferably, in the assist device of the disclosure, when the instep abutting part is configured to include the first portion and the second portion, the instep abutting part includes a third portion connected to the second portion and located on a side opposite that of the first portion in front view. The third portion has a second curved shape convex downward in front view.

With such a configuration, in the wearing state, an end (that is, an end closer to the leg frame than the second portion of the instep abutting part) of the third portion is separated from the upper surface of the foot. Accordingly, it can be prevented that, when the user has the foot turned outward or inward in a direction approaching the leg frame and then turned inward or outward to return, the foot may abut against an end surface of the instep abutting part near the third portion.

Preferably, in the assist device of the disclosure, the locking part includes a pad configured to be locked to the lower leg of the user. The pad has a V shape convex toward a side opposite a side configured to be locked to the user in plan view.

When the force for generating the assist force is applied to the foot from above via the abutting part, since the upper surface of the foot is curved, there is a fear that the abutting part may rotate along the curve due to the force. There is a fear that the locking part that constitutes the second orthosis together with the abutting part may also rotate.

Therefore, when such a pad is provided, a part of the user's leg can be sandwiched by the pad, and the locking part can be locked to the user's leg. Accordingly, the rotation of the locking part (and thus the abutting part that constitutes the second orthosis together with the locking part) can be suppressed.

Preferably, in the assist device of the disclosure, when the locking part is configured to include the pad, the locking part includes a back locking part configured to be locked to a back surface side of the lower leg of the user. The back locking part is configured to be locked to an ankle of the user. The pad is provided on a front surface side of the back locking part.

With such a configuration, the Achilles tendon can be sandwiched and held by the pad. Here, since the Achilles tendon is protruding from a back surface of the lower leg, it can be more easily sandwiched by the pad than a front surface of the lower leg. Accordingly, the rotation of the locking part is further easily suppressed.

Preferably, in the assist device of the disclosure, when the locking part is configured to include the pad, the locking part includes a front locking part configured to be locked to a front surface side of the lower leg of the user, and a back locking part configured to be locked to a back surface side of the lower leg of the user. The front locking part includes, on a back surface side, a front pad configured to be locked to the front surface side of the lower leg of the user. The back locking part includes, on a front surface side, a back pad configured to be locked to the back surface side of the lower leg of the user. At least one of the front pad and the back pad has the V shape convex toward the side opposite the side configured to be locked to the user in plan view.

With such a configuration, the user's leg can be sandwiched from the front and rear by the front locking part and the back locking part. Accordingly, the rotation of the locking part is further easily suppressed.

Preferably, in the assist device of the disclosure, a foot frame extending laterally from the leg frame and rotatably connected to the leg frame about a pitch axis is provided. The second orthosis is connected to the leg frame via the foot frame. The foot frame has a C shape in plan view.

With such a configuration, since the user easily fits the lower leg inside the C-shape foot frame via an opening of the foot frame, the second orthosis connected to the foot frame can be easily worn.

Preferably, in the assist device of the disclosure, the locking part includes a front locking part configured to be locked to a front surface side of the lower leg of the user, and a back locking part configured to be locked to a back surface side of the lower leg of the user. The front locking part is configured to be locked to an ankle of the user. An upper end of the front locking part is located below an upper end of the back locking part.

When the locking part is configured to be locked to the front surface side of the lower leg, when the foot is subjected to dorsiflexion (for example, in the assist force application state), the upper end of the locking part may collide with the user's shin. Therefore, with such a configuration, even if the foot is subjected to plantar flexion, the upper end of the front locking part is less likely to collide with the user's shin.

### First Embodiment

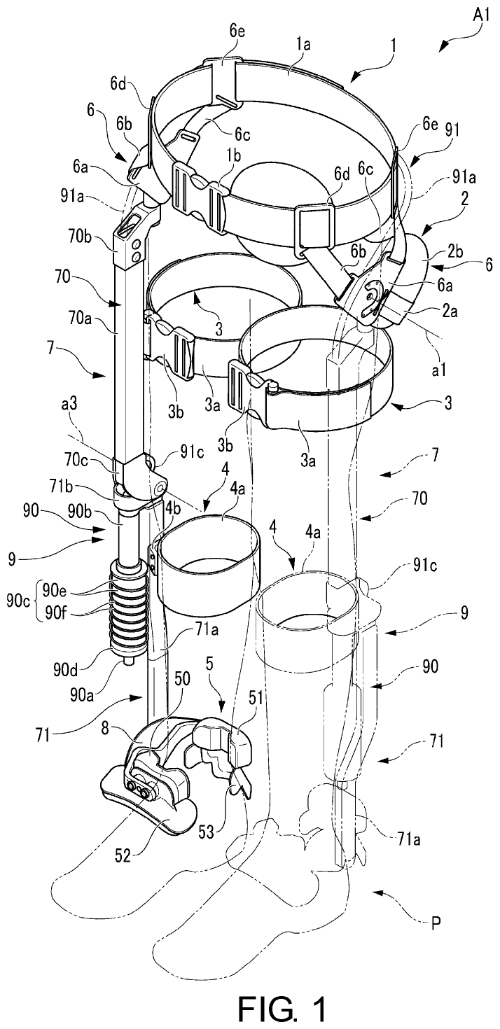

Hereinafter, an assist device A1 according to a first embodiment is described with reference to FIG. 1 to FIG. 9. The assist device A1 is worn on a portion of a user P that ranges from the lumbar region to the ankle, and assists the user P with bending and stretching motions by assisting with the bending and stretching of the leg of the user P. Herein, the bending and stretching motions of the user P include, for example, a motion of the user P when sitting on a chair or the like, a motion when doing a Hindu squat, and the like.

In the following description, a state in which the assist device A1 operates so as to apply an assist force to the body of the user P is referred to as an “assist force application state.” In addition, a state in which the user P wears the assist device A1 is referred to as a “wearing state.”

In the following description, a forward direction of the user P in the wearing state is taken as “front,” and a rearward direction is taken as “rear.” In addition, as viewed from one of a later-described pair of left and right leg frames 7, a side where the other of the leg frames 7 is located is referred to as “inside,” and a side opposite from the other of the leg frames 7 is referred to as “outside.”

First of all, referring to FIG. 1 to FIG. 3, a schematic configuration of the entire assist device A1 is described.

The assist device A1 includes, as orthoses for wearing the assist device A1 on the body of the user P: a lumbar orthosis 1 (first orthosis) worn on the lumbar region; a buttock orthosis 2 (first orthosis) worn on the buttocks; a pair of left and right thigh orthoses 3 respectively worn on the left and right thighs; a pair of left and right lower leg orthoses 4 respectively worn on a portion of the left and right lower legs above the ankle; and a pair of left and right ankle orthoses 5 (second orthosis) respectively worn on the left and right ankles.

In addition, the assist device A1 includes, as members for connecting the orthoses: a pair of left and right support structures 6, hung from both sides of the lumbar orthosis 1 and connected to the buttock orthosis 2 and the thigh orthoses 3; the pair of left and right leg frames 7, each swingably connected to each of the support structures 6 and having the lower leg orthosis 4 attached thereto; and a pair of left and right foot frames 8, each connected to a lower end of each of the leg frames 7 and having the ankle orthosis 5 attached thereto.

In addition, the assist device A1 includes an assist mechanism 9 for, in response to the bending and stretching motions of the leg of the user P, applying the assist force that assists with the bending and stretching motions to the body of the user P by separating the lumbar orthosis 1 and the buttock orthosis 2 from the ankle orthosis 5.

Next, referring to FIG. 1 to FIG. 3, configurations of each portion of the assist device A1 are described.

The lumbar orthosis 1 is worn on the lumbar region (specifically, a portion corresponding to the height of the lumbar vertebrae, such as the upper pelvis, the waist or the like) of the user P. The lumbar orthosis 1 includes: a lumbar belt 1a, formed of a flexible material; and a lumbar buckle 1b, provided at a tip of the lumbar belt 1a.

The lumbar orthosis 1 is worn on the lumbar region of the user P by winding the lumbar belt 1a around the lumbar region of the user P and then connecting the lumbar buckle 1b to form an annular shape. The support structures 6 are suspended on left and right sides of the lumbar belt 1a. The lumbar buckle 1b is provided with a length adjustment mechanism.

The buttock orthosis 2 includes: a buttock belt 2a, provided so as to be bridged between a later-described pair of left and right support plates 6a; a buttock pad 2b, attached to the buttock belt 2a and abutting against the buttocks of the user P; and a pair of left and right connecting belts 2c, suspended from the buttock pad 2b and connected to the corresponding thigh orthoses 3 respectively.

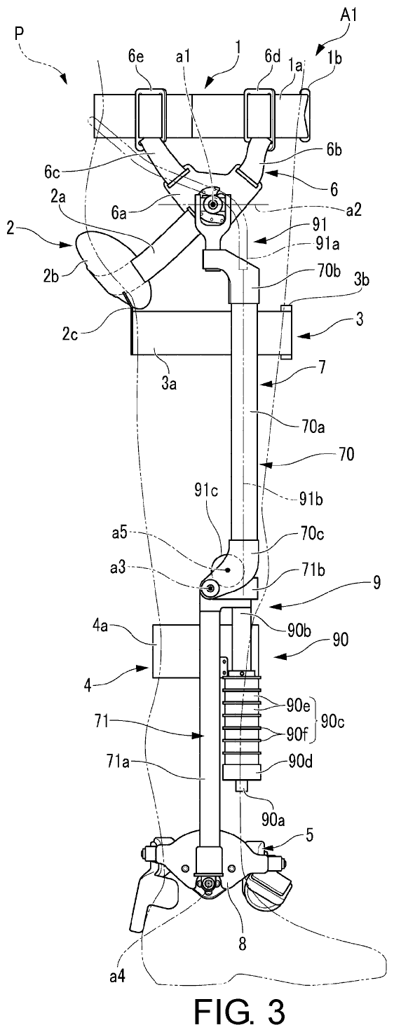

Both ends of the buttock belt 2a are each connected to a rear portion of the corresponding support plate 6a. The buttock belt 2a extends rearward and obliquely downward from the connected portion (see FIG. 3). Accordingly, in the wearing state, the buttock belt 2a is extended in a left-right direction at a lower part of the buttocks of the user P.

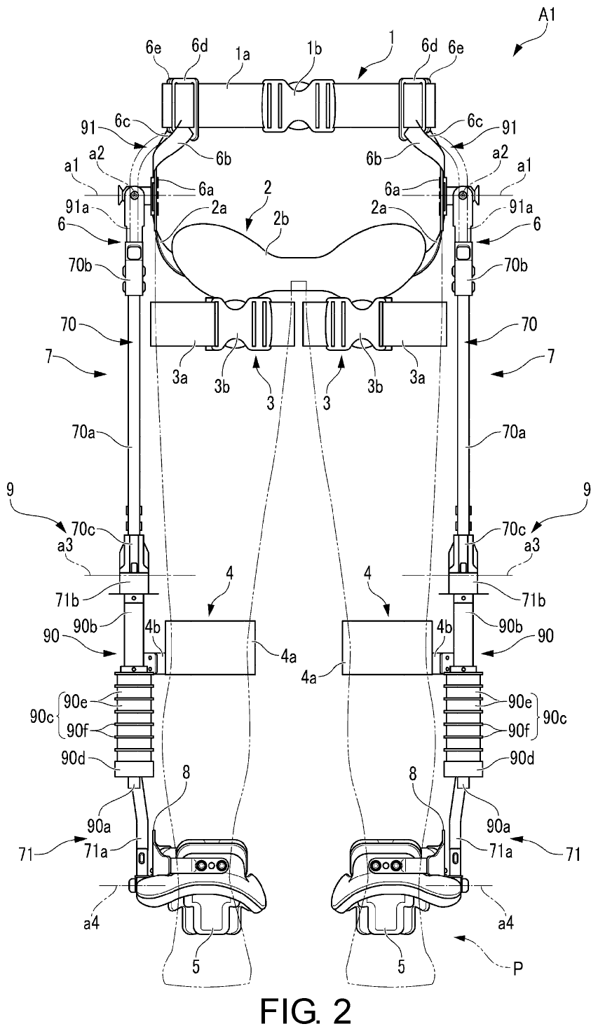

The buttock pad 2b has a gourd-like shape long in the left-right direction and left-right symmetric (see FIG. 2). The buttock pad 2b includes a cushion material inside. In addition, the buttock pad 2b includes an insertion hole penetrating in the left-right direction. The buttock belt 2a is inserted through the insertion hole.

Each of the thigh orthoses 3 is worn on an upper portion of the corresponding thigh of the user P. The thigh orthosis 3 includes: a thigh belt 3a, formed of a flexible material; and a thigh buckle 3b, provided at a tip of the thigh belt 3a.

The thigh orthosis 3 is worn on the thigh of the user P by winding the thigh belt 3a around the thigh of the user P and then connecting the thigh buckle 3b to form an annular shape. The thigh belt 3a is suspended on the buttock pad 2b via the connecting belt 2c. The thigh buckle 3b is provided with a length adjustment mechanism.

Each of the lower leg orthoses 4 is worn on an upper portion (specifically, an upper part of the calf) of the corresponding lower leg of the user P. The lower leg orthosis 4 includes: a lower leg belt 4a, formed of a flexible material; a connecting fitting 4b, for connection to a later-described lower leg frame member 71; and a surface fastener (not shown).

The lower leg orthosis 4 is worn on the lower leg of the user P by winding the lower leg belt 4a and fixing the wound lower leg belt 4a with the surface fastener. In the lower leg orthosis 4, the surface fastener also functions as a length adjustment mechanism. The lower leg orthosis 4 is connected to the later-described lower leg frame member 71 via the connecting fitting 4b.

Each of the ankle orthoses 5 includes a pair of front and rear members, and is worn on the front and rear of the corresponding ankle of the user P. In a state (hereinafter “assist force application state”) in which the assist device A1 operates so as to apply the assist force to the body of the user P, the ankle orthosis 5 abuts at its lower end against an upper surface (for example, an instep, and an upper surface of a portion inclined forward above a heel) of the foot of the user P.

Each of the support structures 6 faces a side surface of the lumbar region of the user P in the wearing state. Each of the support structures 6 includes: each of the pair of left and right support plates 6a, swingably supporting the corresponding leg frame 7; a front hanging belt 6b, extending forward and obliquely upward from the support plates 6, a rear hanging belt 6c, extending rearward and obliquely upward from the support plate 6a; a front attachment fitting 6d, provided at a tip of the front hanging belt 6b; and a rear attachment fitting 6e, provided at a tip of the rear hanging belt 6c.

The support plate 6a is formed of a highly rigid material such as metal or the like. The support plate 6a has a substantially pentagonal shape. The front hanging belt 6b is connected to a front portion of the support plate 6a, and the rear hanging belt 6c is connected to a rear portion of the support plate 6a.

In addition, a tip of the buttock belt 2a is connected to a rear part of the support plate 6a below the connected portion with the rear hanging belt 6c. The buttock belt 2a extends rearward and obliquely downward from the support plate 6a. Accordingly, the buttock belt 2a is extended on an extension line of the front hanging belt 6b with the support plate 6a interposed therebetween.

The front hanging belt 6b is connected to a front portion of the lumbar belt 1a via the front attachment fitting 6d. In addition, the rear hanging belt 6c is connected to a rear portion of the lumbar belt 1a via the rear attachment fitting 6e.

Here, the front attachment fitting 6d is provided with a length adjustment mechanism of the front hanging belt 6b, and the rear attachment fitting 6e is provided with a length adjustment mechanism of the rear hanging belt 6c. By these length adjustment mechanisms, a position (that is, positions and heights of later-described first axis a1 and second axis a2) of the support plate 6a in the wearing state is adjusted.

Each of the leg frames 7 includes: a thigh frame member 70, extending in the up-down direction outside the corresponding thigh of the user P in the wearing state; and the lower leg frame member 71, extending in the up-down direction outside the corresponding lower leg of the user P in the wearing state.

The thigh frame member 70 includes: a first body 70a of a tubular shape, extending in the up-down direction; a first connector 70b, extending rearward and obliquely upward from an upper end of the first body 70a; and a second connector 70c, extending rearward and obliquely downward from a lower end of the first body 70a.

The first connector 70b is swingably connected at its tip (rear portion) to the support plate 6a about the first axis a1 being an axis extending in a pitch axis direction and about the second axis a2 being an axis extending in a roll axis direction. In addition, the first connector 70b includes, at its base end (front portion), a through hole (not shown) penetrating the first connector 70b in the up-down direction and connected to the inside of the first body 70a.

When the user P wears the assist device A, the positions of the first axis a1 and the second axis a2 are adjusted by the front hanging belt 6b, the rear hanging belt 6c, the front attachment fitting 6d and the rear attachment fitting 6e of the support structure 6 so as to pass through the center of a hip joint of the user P.

The second connector 70c is swingably connected at its tip (rear portion) to a later-described third connector 71b about a third axis a3 being an axis extending in the pitch axis direction. In addition, the second connector 70c includes, at its base end (front portion), a through hole (not shown) penetrating the second connector 70c in the up-down direction and connected to the inside of the first body 70a.

In the thigh frame member 70, a later-described wire 91b is slidably inserted through the inside of the first body 70a, the through hole of the first connector 70b and the through hole of the second connector 70c.

The lower leg frame member 71 includes: a second body 71a, extending in the up-down direction; and the third connector 71b, extending forward and obliquely upward from an upper end of the second body 71a.

The third connector 71b is swingably connected at its base end (rear portion) to the second connector 70c about the third axis a3. In addition, the third connector 71b includes, at its tip (front portion), a through hole (not shown) penetrating the third connector 71b in the up-down direction and connected to the inside of a later-described guide pipe 90a.

In the wearing state, the third axis a3 is in a position passing through the center of a knee joint of the user P. The position of the third axis a3 may be configured to be adjustable by provision of a length adjustment mechanism on the thigh frame member 70, or the like.

In the wearing state, the leg frame 7 configured in this way operates integrally with the leg of the user P so as to be taken along by the leg of the user P via the lower leg orthosis 4 and the foot frame 8 connected to the lower leg frame member 71.

Specifically, in response to swinging of each thigh with respect to the body of the user P, the thigh frame member 70 corresponding to each thigh swings about the first axis a1 or the second axis a2. Accordingly, a positional relationship between the thigh frame member 70 and the thigh of the user P is maintained substantially constant.

In addition, in response to swinging of each lower leg corresponding to each thigh of the user P, the lower leg frame member 71 corresponding to each lower leg swings about the third axis a3. Accordingly, a positional relationship between the lower leg frame member 71 and the lower leg of the user P is maintained substantially constant.

Each of the foot frames 8 is swingably connected to a lower end of the second body 71a of the corresponding lower leg frame member 71 about a fourth axis a4 being an axis extending in the pitch axis direction. In addition, each of the foot frames 8 holds the ankle orthosis 5 including the pair of front and rear members.

The assist mechanism 9 is composed of a pair of left and right tension generators 90 provided on each leg frame 7 and a transmission mechanism 91 for transmitting a force generated by the tension generator 90.

The tension generator 90 includes: the guide pipe 90a, extending downward from the tip (front portion) of the third connector 71b of the lower leg frame member 71; an adjustment pipe 90b, through which a portion on a base end side (near the third connector 71b) of the guide pipe 90a is inserted; an elastic member 90c of a tubular shape, through which a portion on a tip side of the guide pipe 90a is inserted; an end member 90d, slidably attached to the tip of the guide pipe 90a in an axial direction of the guide pipe 90a; and a coil spring (not shown), disposed inside the guide pipe 90a.

The later-described wire 91b is inserted through the guide pipe 90a. The wire 91b led out from the guide pipe 90a passes through the through hole of the third connector 71b and is led out above the third connector 71b. A plurality of slots extending in the axial direction are formed in the guide pipe 90a.

The guide pipe 90a extends downward from the tip (front portion) of the third connector 71b of the lower leg frame member 71. Accordingly, the guide pipe 90a (and thus the tension generator 90) is disposed along a front surface of the lower leg frame member 71, and is located on the axis of the thigh frame member 70 if the user P in the wearing state is standing (see FIG. 3).

The adjustment pipe 90b is in abutting contact at its upper end with the third connector 71b, and is in abutting contact at its lower end with the elastic member 90c. The length of the adjustment pipe 90b can be adjusted by cutting. Moreover, the length of the adjustment pipe 90b may be configured to be reversibly adjustable by provision of an expansion mechanism or the like.

By adjusting the length of the adjustment pipe 90b, a length of a tubular member composed of the adjustment pipe 90b and the elastic member 90c is adjusted. Specifically, the length is adjusted so that, when the user P in the wearing state is standing, the elastic member 90c does not rattle and is not compressed because it is supported by the end member 90d.

The elastic member 90c is a multi-layered tubular member configured by alternately superimposing a plurality of elastic bodies 90e of a tubular shape and a plurality of plates 90f of an annular shape. The elastic member 90c is externally mounted on an outer periphery of the guide pipe 90a so as to be expandable and contractible in the axial direction.

Each of the elastic bodies 90e is formed of a material having a large number of built-in sealed air cells. Specifically, each of the elastic bodies 90e is formed of, for example, a single-cell (closed-cell) rubber sponge or the like. The elastic body 90e has an elliptical shape as viewed from the axial direction in an uncompressed state (natural state).

Each of the plates 90f is formed of a material having higher rigidity than the material forming the elastic body 90e. Specifically, each of the plates 90f is formed of, for example, metal, hard resin or the like. The plate 90f has an elliptical shape as viewed from the axial direction. The elliptical shape is of a size so as to overlap the entire elastic body 90e as viewed in the axial direction when the plate 90f is laminated on the elastic body 90.

The elastic member 90c is configured by alternately laminating the elastic body 90e and the plate 90f and fixing them to each other with an adhesive or the like. Here, the elastic member 90c abuts at its upper end against the adjustment pipe 90b, and abuts at its lower end against the end member 90d. Hence, the elastic member 90c is configured so that the plate 90f is located at both ends in the up-down direction in order to form the abutting surfaces.

The elastic member 90c configured in this way is less likely to buckle or bend and is lighter as compared with a metal spring or the like that generates a similar elastic force. However, the elastic member does not necessarily have to be composed of an elastic body and a plate, and may be anything capable of generating an elastic force. Hence, the configuration of the elastic member may be appropriately changed depending on the performance of other components of the assist device, or the like. For example, a coil spring may be used as the elastic member.

The end member 90d is formed as an annular member. A plurality of pins (not shown) protruding inward are provided on an inner peripheral surface of the end member 90d. By inserting each of the pins into the corresponding slot of the guide pipe 90a, the end member 90d is slidably attached to the guide pipe 90a in the axial direction.

The end member 90d is in abutting contact with the lower end of the elastic member 90c. In addition, the end member 90d is connected to the wire 91b inserted through the inside of the guide pipe 90a. Accordingly, the end member 90d compresses the elastic member 90c to generate the elastic force while sliding with respect to the guide pipe 90a in response to advance or retreat of the wire 91b.

Inside the guide pipe 90a, the coil spring (not shown) is in abutting contact at its upper end with the third connector 71b, and is in abutting contact at its lower end with an upper surface of the end member 90d. The coil spring energizes the end member 90d in an extension direction of the elastic member 90c. Accordingly, a weak tension (pre-tension) that eliminates looseness is applied via the end member 90d to the wire 91b connected to the end member 90d.

Rigidity (that is, spring constant) of the coil spring is sufficiently smaller than rigidity of the elastic member 90c. Specifically, the rigidity of the coil spring is large enough to allow the user P to bend the knee joint during walking or the like without feeling discomfort.

The transmission mechanism 91 includes: an outer tube 91a, crossing a back surface of the lumbar orthosis 1 and connected to the first connector 70b of the left and right thigh frame members 70; the wire 91b, slidably inserted through the outer tube 91a; and a pulley 91c, provided on an upper surface of the third connector 71b of the lower leg frame member 71 and on which the wire 91b is hung.

Both ends of the outer tube 91a are each connected to the through hole of the corresponding first connector 70b. Accordingly, a space inside the outer tube 91a, the through hole of the first connector 70b of the thigh frame member 70, a space inside the first body 70a, and the through hole of the second connector 70c are in communication with each other.

The wire 91b is slidably inserted through the outer tube 91a. A central part of the wire 91b is slidably inserted into the outer tube 91a and the thigh frame member 70. Both ends of the wire 91b are each led out from a lower end of the through hole of the second connector 70c of the corresponding thigh frame member 70, and introduced into the guide pipe 90a via the through hole of the third connector 71b of the lower leg frame member 71.

Both ends of the wire 91b are each connected to the end member 90d inside the guide pipe 90a. Hence, the elastic force generated by the elastic member 90c is applied to both ends of the wire 91b via the end member 90d. That is, the elastic force generated by the elastic member 90c acts as a tensile force on the wire 91b.

A portion of the wire 91b led out from the lower end of the through hole of the second connector 70c and introduced into an upper end of the through hole of the third connector 71b is hung on the pulley 91c. The pulley 91c is rotatable about a fifth axis a5 being an axis extending in the pitch axis direction.

When the user P in the wearing state is standing (see FIG. 3), an apex on the front side of a peripheral surface of the pulley 91c is located on a straight line connecting the lower end of the through hole of the second connector 70c and the upper end of the through hole of the third connector 71b.

Hence, in this state, since the pulley 91c is only in slight contact with the wire 91b, a path length of the portion of the wire 91b between the lower end of the second connector 70c and the upper end of the third connector 71b is not changed.

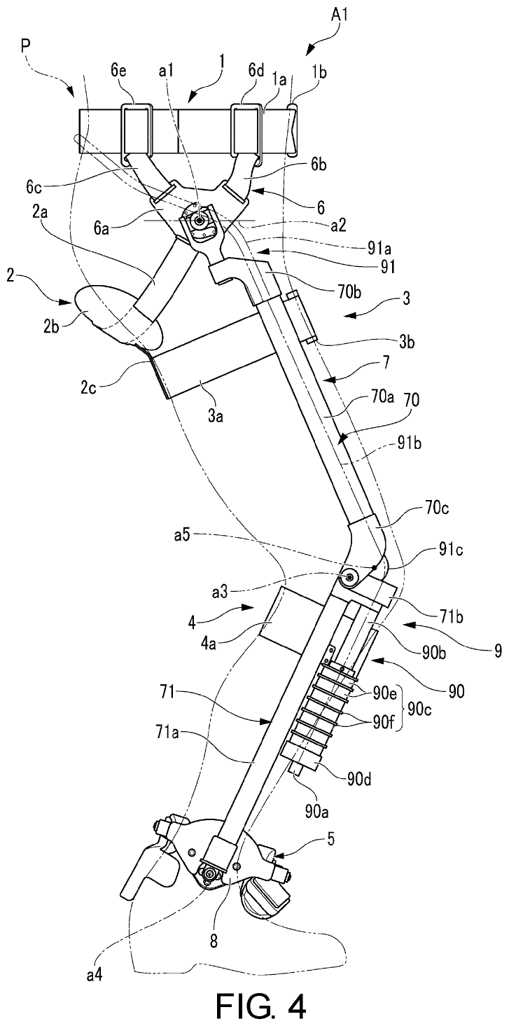

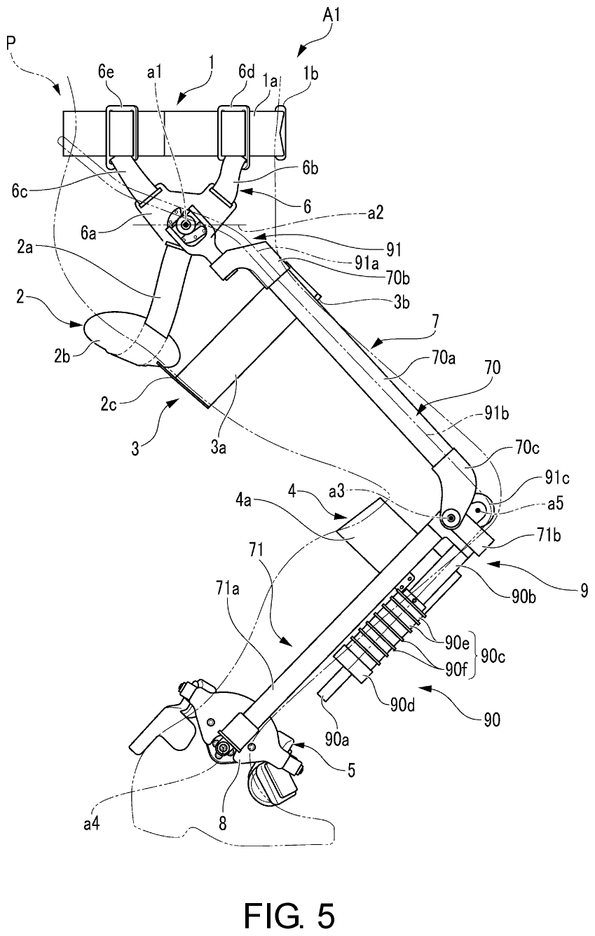

Here, the fifth axis a5 being a rotation axis of the pulley 91c is located above and in front of the third axis a3 corresponding to the center of the knee joint of the user P. Hence, when the user P in the wearing state has bent the knee (see FIG. 4 and FIG. 5), in response to the bending of the knee of the user P, the apex on the front side of the peripheral surface of the pulley 91c moves to a position in front of the straight line connecting the lower end of the through hole of the second connector 70c and the upper end of the through hole of the third connector 71b.

Hence, in the state in which the user P in the wearing state has bent the knee, the pulley 91c pushes the wire 91b forward and increases the path length of the portion of the wire 91b between the thigh frame member 70 and the third connector 71b.

In this way, by the pulley 91c, the path length of the wire 91b between the lower end of the second connector 70c and the third connector 71b increases as a bending degree (that is, rotation degree of the knee joint) of the leg of the user P in the wearing state increases.

That is, the pulley 91c functions as a cam member changing the path of the wire 91b so that the path length of the wire 91b increases as a swing angle of the thigh frame member 70 with respect to the lower leg frame member 71 increases.

Next, referring to FIG. 3 to FIG. 5, an operation of the assist device A1 when applying the assist force to the body of the user P is described.

At a stage where the posture of the user P transits from the standing state (state shown in FIG. 3) to the state in which the knee is bent (state shown in FIG. 4 or FIG. 5), the bending degree of the knee joint of the user P gradually increases. Meanwhile, as the knee joint of the user P is bent (that is, the lower leg swings with respect to the thigh), the lower leg frame member 71 swings with respect to the thigh frame member 70.

At this time, the pulley 91c moves as the bending degree increases, and the path length of the wire 91b hung on the pulley 91c increases. Accordingly, the end member 90d connected to a tip of the wire 91b begins to move in a direction of compressing the elastic member 90c.

However, as shown in FIG. 4, at a stage where the knee is only slightly bent, the movement of the end member 90d is suppressed by the energizing force of the coil spring disposed inside the guide pipe 90a, and the elastic member 90c is hardly compressed.

That is, at this stage, the elastic force (that is, the tensile force applied to the wire 91b) generated by the elastic member 90c also remains sufficiently small. Hence, at this stage, the assist force that assists with the bending and stretching motions is hardly applied to the body of the user P.

On the other hand, as shown in FIG. 5, at a stage where the knee is largely bent, the movement of the end member 90d can no longer be suppressed by the energizing force of the coil spring disposed inside the guide pipe 90a, the elastic member 90c is compressed and the elastic force begins to be generated.

That is, at this stage, the elastic force (that is, the tensile force applied to the wire 91b) generated by the elastic member 90c also becomes sufficiently large. Hence, at this stage, the assist force that assists with the bending and stretching motions is applied to the body of the user P.

Specifically, the elastic force generated by the elastic member 90c becomes the tensile force of the wire 91b. The tensile force acts, about the third axis a3, as a force (force in a direction of returning from the state shown in FIG. 5 to the state shown in FIG. 3) in a direction of returning the thigh frame member 70 through which the wire 91b is inserted and the lower leg frame member 71 in which the wire 91b is fixed via the guide pipe 90a to an initial state.

With respect to the body of the user P, the above force acts as the assist force separating the lumbar region and the buttocks from the ankle of the user P (that is, assisting with the stretching motion at the knee joint) via the lumbar orthosis 1 and the buttock orthosis 2 connected to the thigh frame member 70 via the support structure 6 and the ankle orthosis 5 connected to the lower leg frame member 71 via the foot frame 8.

By the way, unlike the conventional assist device as described in Patent Document 1, in the assist device A1, as an orthosis worn on the foot, the ankle orthosis 5 worn on the ankle of the user P, instead of the type that carries the foot of the user P, is adopted.

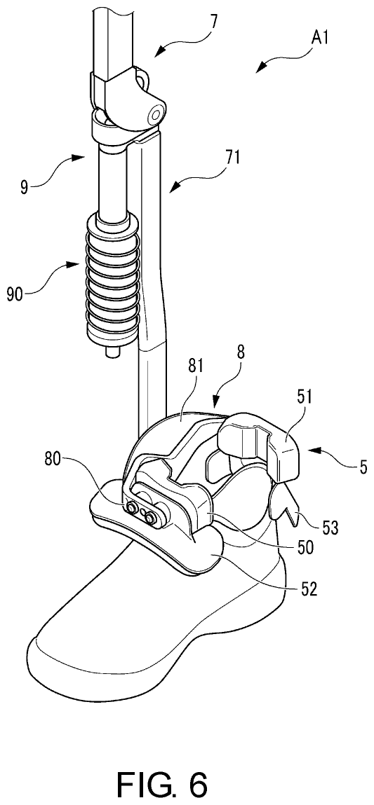

Therefore, in the following, configurations of the ankle orthosis 5 of the assist device A1 and the foot frame 8 connecting the ankle orthosis 5 to the lower leg frame member 71 of the leg frame 7 are described in detail with reference to FIG. 6 to FIG. 8.

First of all, referring to FIG. 6 to FIG. 9, a schematic configuration of the ankle orthosis 5 and the foot frame 8 and a configuration of a connected portion between the ankle orthosis 5 and the foot frame 8 are described.

As shown in FIG. 6, the ankle orthosis 5 includes: a front locking part 50, configured to be locked on a front surface side of the ankle of the user P in the wearing state; and a back locking part 51, configured to be locked on a back surface side of the ankle.

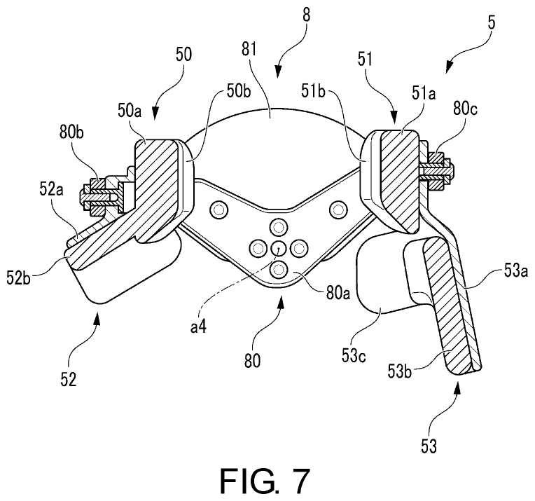

As shown in FIG. 7, an upper end of the front locking part 50 is located below an upper end of the back locking part 51. Accordingly, when the user P has the foot dorsiflexed in the wearing state (for example, in the assist force application state (see FIG. 4 and FIG. 5)), the upper end of the front locking part 50 is less likely to collide with the shin of the user P.

In addition, the ankle orthosis 5 includes: an instep abutting part 52, abutting against the instep of the user P in the assist force application state; and a heel abutting part 53, abutting against a portion on a back surface side of the foot of the user P that is inclined forward above the heel in the assist force application state. Moreover, the instep abutting part 52 and the heel abutting part 53 may abut against the instep of the user P and the portion on the back surface side of the foot of the user P that is inclined forward above the heel via a shoe, a sock or the like worn by the user P.

The heel abutting part of the disclosure is not limited to such a configuration, and may be anything that abuts against the user's foot or ankle from the heel side. Hence, for example, if a locking part is configured to be locked at a relatively high position below the knee joint, such as the calf or the like, the heel abutting part may be configured to abut against the ankle instead of the foot from the back surface side.

In the assist force application state, a force generated by the elastic member 90c and transmitted from the lower leg frame member 71 acts on the instep of the user P via the instep abutting part 52. In addition, in the assist force application state, a force generated by the elastic member 90c and transmitted from the lower leg frame member 71 acts on the portion inclined forward above the heel of the user P via the heel abutting part 53.

The foot frame 8 includes: a foot frame member 80, rotatably connected to a lower end of the lower leg frame member 71 about the fourth axis a4; and a protective plate 81, fixed to the outside of the foot frame member 80 and for preventing the lower leg of the user P from contacting the lower leg frame member 71 in the wearing state.

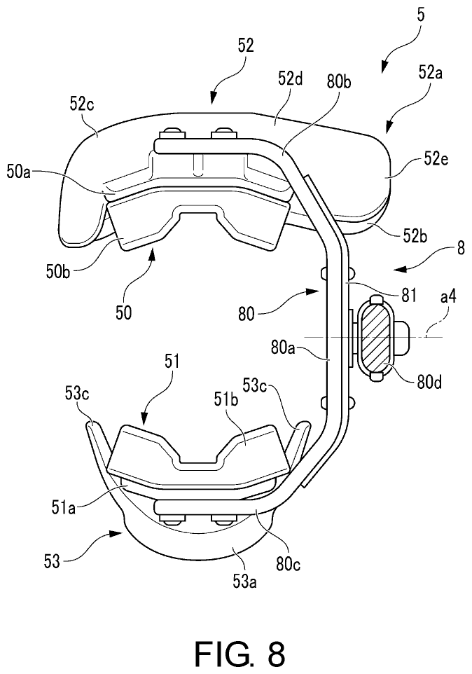

As shown in FIG. 8, the foot frame member 80 includes: an intermediate 80a, connected to the lower leg frame member 71; a front part 80b, extending inward from a front end of the intermediate 80a; and a rear part 80c, extending inward from a rear end of the intermediate 80a.

The intermediate 80a is curved inward in plan view. That is, the foot frame member 80 has a C shape in plan view. In addition, the front locking part 50 is held on a rear side of the front part 80b, and the back locking part 51 is held on a front side of the rear part 80c.

Since the foot frame member 80 is configured in this way, the user P is able to fit the ankle inside the foot frame member 80 via an opening (that is, a portion facing the intermediate 80a) of the C shape. Accordingly, in the assist device A1, the front locking part 50 and the back locking part 51 (and thus the ankle orthosis 5) can be easily mounted.

By the way, the assist device A1 is configured to apply the assist force to the body of the user P by swinging the lower leg frame member 71 with respect to the thigh frame member 70 and separating the lumbar orthosis 1 and the buttock orthosis 2 from the ankle orthosis 5. In the case of such a configuration, in the assist force application state, the force is transmitted from the lower leg frame member 71 to the instep abutting part 52 and the heel abutting part 53.

At this time, the greater the distance from a connected portion (specifically, a later-described fourth connector 80d) between the foot frame 8 (and thus the instep abutting part 52 and the heel abutting part 53) and the lower leg frame member 71 to the instep abutting part 52 and the heel abutting part 53, the greater the force applied from above from the instep abutting part 52 and the heel abutting part 53 to the foot of the user P. Thus, due to the abutting contact with the instep abutting part 52 and the heel abutting part 53, the discomfort felt in the foot by the user P is also increased.

In order to suppress this discomfort, the lower leg frame member 71 is preferably directly connected to the instep abutting part 52 and the heel abutting part 53. However, in the structure of the assist device A1, due to the shape of the lower leg frame member 71, the arrangement of the tension generator 90, and the like, it is difficult to directly connect the lower leg frame member 71 to the instep abutting part 52 and the heel abutting part 53.

Hence, in the assist device A1, the lower leg frame member 71 is connected to the instep abutting part 52 and the heel abutting part 53 via the front locking part 50, the back locking part 51 and the foot frame 8.

Here, as shown in FIG. 7, the instep abutting part 52 extends forward and obliquely downward from a lower portion of the front locking part 50. The heel abutting part 53 extends rearward and obliquely downward from a lower portion of the back locking part 51. With such a configuration, in the assist device A1, the distance from the connected portion between the foot frame 8 and the lower leg frame member 71 to the instep abutting part 52 and the heel abutting part 53 is made as small as possible.

Accordingly, in the assist force application state, the force applied from above from the instep abutting part 52 and the heel abutting part 53 to the foot of the user P is reduced, and the discomfort felt in the foot by the user P due to the abutting contact with the instep abutting part 52 and the heel abutting part 53 is suppressed.

Accordingly, a member for connecting the instep abutting part 52 and the front locking part 50 as well as a member for connecting the heel abutting part 53 and the back locking part 51 are omitted, and the size, weight and cost are reduced.

By the way, when a force for generating the assist force is applied from the lower leg frame member 71 via the instep abutting part 52 and the heel abutting part 53 to the instep and heel of the user P, a force in the axial direction of the lower leg frame member 71 is applied from the lower leg frame member 71 to the foot frame member 80. For example, when the state shown in FIG. 3 is transited to the state shown in FIG. 4, a downward force is applied from the lower leg frame member 71 toward the foot frame member 80.

Here, as shown in FIG. 6, the foot frame member 80 has a C shape in plan view. That is, the front locking part 50 attached to the front part 80b of the foot frame member 80 and the back locking part 51 attached to the rear part 80c of the foot frame member 80 are located laterally with respect to the lower leg frame member 71 connected to the intermediate 80a.

Hence, due to the force applied from the lower leg frame member 71 to the foot frame member 80, there is a fear that the instep abutting part 52 and the heel abutting part 53 may rotate about the roll axis along the curve of the upper surface of the foot of the user P.

Therefore, as shown in FIG. 7 and FIG. 8, in the assist device A1, the front locking part 50 is fixed to the front part 80b of the foot frame member 80, and the back locking part 51 is fixed to the rear part 80c of the foot frame member 80. That is, with respect to the lower leg frame member 71 to which the foot frame member 80 is attached, the instep abutting part 52 and the heel abutting part 53 are configured to be non-rotatable about the roll axis. Accordingly, unintended rotation of the instep abutting part 52 and the heel abutting part 53 about the roll axis is suppressed.

Moreover, in the conventional assist device adopting the type that carries the foot as the foot orthosis as described in Patent Document 1, in order to prevent the motion of the ankle joint of the user P from being hindered in the wearing state, it is necessary to allow an abutting part of the foot orthosis to rotate about the roll axis. Thus, it is necessary to provide a connecting mechanism for allowing such rotation about the roll axis.

However, in the assist device A1, since the ankle orthosis 5 locked to the ankle is adopted as the foot orthosis, even if the rotation of the instep abutting part 52 and the heel abutting part 53 being the abutting part of the ankle orthosis 5 is configured not to be allowed, the motion of the ankle joint of the user P will not be hindered.

Hence, since the connecting mechanism for allowing the rotation about the roll axis can be omitted from the assist device A1, the size, weight and cost can be reduced as compared with the conventional assist device.

Moreover, the assist device of the disclosure is not necessarily limited to the configuration in which the abutting part is non-rotatable about the roll axis. For example, if the abutting part is not provided with a pad as described later, in order to absorb the impact when the user's foot abuts against the abutting part, the abutting part may be slightly rotatable about the roll axis.

As shown in FIG. 7, the intermediate 80a of the foot frame member 80 has a V shape convex downward in side view. The fourth connector 80d being a connected portion (specifically, the fourth axis a4) with respect to the lower leg frame member 71 is provided in a central part (the lowest portion).

Accordingly, the fourth connector 80d is located below a later-described front pad 50b of the front locking part 50 and a later-described back pad 51b of the back locking part 51 (that is, a locking position of the front locking part 50 and a locking position of the back locking part 51 with respect to the ankle of the user P).

With such a configuration, in the wearing state, the fourth connector 80d, which rotatably connects the lower leg frame member 71 and the foot frame member 80 about the fourth axis a4 being the pitch axis, is located close to the ankle of the user P in the up-down direction.

In addition, the fourth connector 80d (strictly speaking, the fourth axis a4) that connects the foot frame member 80 to the lower leg frame member 71 is located between the later-described front pad 50b of the front locking part 50 and the later-described back pad 51 of the back locking part 51 (that is, between the locking position of the front locking part 50 and the locking position of the back locking part 51 with respect to the ankle of the user P).

With such a configuration, in the wearing state, the fourth connector 80d connecting the lower leg frame member 71 and the foot frame member 80 is located close to the ankle of the user P in the front-rear direction.

With these configurations, in the assist device A1, a difference between the motion of the foot frame member 80 (and thus the instep abutting part 52 and the heel abutting part 53 that apply force to the user P) and the motion of the foot of the user P is reduced.

As a result, in the assist device A1, a deviation of the instep abutting part 52 and the heel abutting part 53 from the foot of the user P is suppressed, and a shift in the COP of the user P in the assist force application state can also be reduced. Thus, the COP is easily kept within a range of a support base surface.

In addition, in the present embodiment, in the wearing state, the fourth connector 80d being the connected portion between the lower leg frame member 71 of the leg frame 7 and the foot frame member 80 of the foot frame 8 is provided in a position corresponding to the ankle of the user P in this way. However, the position of the connecting member of the disclosure is not limited to such a configuration, and may be appropriately determined according to the shape of other components of the assist device and the like.

Hence, for example, like a later-described assist device A2 of the second embodiment, in the wearing state, the connected portion may be configured to be located in front of the ankle of the user P.

Next, the components of the ankle orthosis 5 are described in detail with reference to FIG. 7 to FIG. 9.

When the force for generating the assist force is applied to the foot of the user P from above via the instep abutting part 52 and the heel abutting part 53, since the upper surface of the foot is curved, there is a fear that the instep abutting part 52 and the heel abutting part 53 may rotate due to the force. Then, there is a fear that the front locking part 50 and the back locking part 51 may also rotate together with the instep abutting part 52 and the heel abutting part 53.

Therefore, as shown in FIG. 7, in the front locking part 50, on a back surface side of the front body 50a connected to the foot frame member 80, the front pad 50b configured to be locked to the front surface side of the ankle of the user P is provided. In addition, in the back locking part 51, on a front surface side of the front body 51a connected to the foot frame member 80, the back pad 51b configured to be locked to the back surface side of the ankle of the user P is provided.

In the wearing state, the front locking part 50 and the back locking part 51 are locked to the ankle so that the ankle of the user P is sandwiched from the front and rear by the front pad 50b and the back pad 51b.

Here, as shown in FIG. 8, the front pad 50b has a V shape convex forward. The back pad 51b has a V shape convex rearward.

Accordingly, the front pad 50b is able to sandwich and hold a front surface side of the base of the foot. In addition, the back pad 51b is able to sandwich and hold the Achilles tendon protruding from the back surface of the lower leg.

With these configurations, in the assist device A1, the rotation of the front locking part 50 and the back locking part 51 is able to be suppressed. Thus, the instep abutting part 52 and the heel abutting part 53, which constitute the ankle orthosis 5 together with the front locking part 50 and the back locking part 51, are able to be suppressed from rotating.

Moreover, the instep abutting part 52 includes, on a lower surface side of an instep-side body 52a connected to the front body 50a, an instep pad 52b abutting against the instep of the user P. The heel abutting part 53 includes, on a front surface side of a heel-side body 53a connected to the back body 51a, a heel pad 53b abutting against the portion inclined forward above the heel of the user P.

The instep pad 52b and the heel pad 53b are buffer members for absorbing the impact when a force is applied to the upper surface of the foot of the user P in the assist force application state.

Moreover, in the present embodiment, the ankle orthosis 5 includes the front pad 50b, the back pad 51b, the instep pad 52b and the heel pad 53b that have a V shape in plan view in this way. However, the pad of the disclosure is not limited to such number and shape.

Hence, for example, the number of the pad may be three or less or may be five or more. Also, the pad may be omitted. The shape of the pad may be a flat shape instead of a V shape in plan view.

In addition, as shown in FIG. 7 and FIG. 8, the heel abutting part 53 includes a pair of left and right collars 53c extending forward from both sides thereof. Each of the collars 53c faces the corresponding side surface of the heel of the user P in the wearing state.

In general, when the user P moves the foot about the ankle joint, the amount of movement of the rearfoot (portion on the heel side) is smaller than the amount of movement of the forefoot (portion on the instep side). Hence, when the collar 53c like this is provided on the heel abutting part 53 abutting against the foot of the user P from the heel side (that is, abutting against the rearfoot from the back surface side), even when the user P moves the foot in the wearing state, a state is maintained in which both sides of the heel of the user P are in stable abutting contact with the collar 53c.

If both sides of the heel of the user P are in abutting contact with the collar 53c, due to the abutting contact, the rotation of the heel abutting part 53 at the time when the force for generating the assist force is applied to the portion inclined forward above the heel of the user P can be suppressed.

Moreover, in the present embodiment, the pair of left and right collars 53c extend forward from both sides of the heel abutting part 53 in this way. However, the collar of the disclosure may be anything that extends from the heel abutting part so as to be located on a side surface of the foot of the user in the wearing state.

Hence, for example, there may be provided only one collar, or three or more collars. Also, the collar may be omitted. The collar may be provided on the instep abutting part. Here, if the collar is provided on the instep abutting part, the collar may extend downward.

By the way, when the instep abutting part 52 is rotated by the force for generating the assist force, since the instep is curved so as to be convex upward in front view, there is a fear that the instep abutting part 52 may rotate about the roll axis. Since the instep is inclined toward a toe side, there is also a fear that the instep abutting part 52 may rotate about a yaw axis. There is also a fear that the instep abutting part 52 may be attracted to the lower leg frame member 71 due to the force.

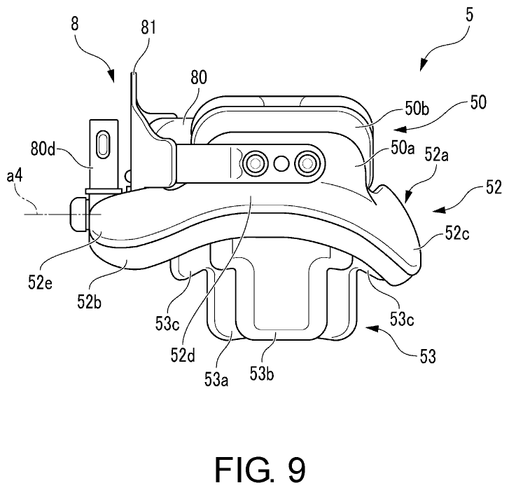

Therefore, as shown in FIG. 9, the instep-side body 52a of the instep abutting part 52 is composed of: a first portion 52c, located inward from the fourth connector 80d (and thus the lower leg frame member 71 connected to the fourth connector 80d) in front view; a second portion 52d, connected to the first portion 52c and located close to the fourth connector 80d in front view; and a third portion 52e, connected to the second portion 52d and located on a side opposite that of the first portion 52c in front view.

The first portion 52c and the second portion 52d form a first curved shape convex upward in front view. Specifically, the first portion 52c constitutes a portion on a side farther from the lower leg frame member 71 than an apex of the first curved shape, and the second portion 52d constitutes a portion closer to the lower leg frame member 71 than the apex. In addition, the third portion 52e has a second curved shape convex downward in front view.

The instep pad 52b provided on the lower surface side of the instep-side body 52a also has a curved shape similar to that of the instep-side body 52a.

In the wearing state, the instep abutting part 52 abuts against the foot of the user P at the first portion 52c and the second portion 52d. The first portion 52c and the second portion 52d have the first curved shape convex upward in front view. Accordingly, since the first portion 52c and the second portion 52d firmly abut against the instep of the user P in the assist force application state, the rotation of the instep abutting part 52 is suppressed.

Here, a radius of curvature of the first portion 52c is set smaller than a radius of curvature of the second portion 52d. Hence, the first portion 52c is located on a lower side (that is, near the side surface of the foot of the user P in the wearing state) as compared with the second portion 52d. In the wearing state, the first portion 52c is located opposite the fourth connector 80d (and thus the lower leg frame member 71) with the foot of the user P sandwiched therebetween.

Accordingly, even if the instep abutting part 52 is attracted to the lower leg frame member 71 by the force for generating the assist force, the first portion 52c is brought into abutting contact with the side surface of the foot of the user P and it can be prevented that the instep abutting part 52 may move to be attracted.

In the wearing state, the third portion 52e of the instep abutting part 52 is located outside the foot of the user P in front view. The third portion 52e has the second curved shape convex downward. Hence, in the wearing state, an end (that is, an end closer to the lower leg frame member 71 than the second portion 52d of the instep abutting part 52) of the third portion 52e is separated from the upper surface of the foot.

Accordingly, it is prevented that, when the user P has the foot turned outward in a direction approaching the lower leg frame member 71 and then turned inward to return, the foot may abut against an end surface of the instep abutting part 52 near the third portion 52e.

In the present embodiment, the instep abutting part 52 is composed of the instep-side body 52a and the instep pad 52b, the instep-side body 52a being composed of the first portion 52c, the second portion 52d and the third portion 52e. However, the instep abutting part of the disclosure is not limited to such a configuration.

Hence, for example, the shape of the instep abutting part may not be a shape including the first curved shape and the second curved shape, but may be a shape including only the first curved shape, or may simply be a flat plate shape.

As described above, in the assist device A1, in the assist force application state, the ankle orthosis 5 abuts against the foot of the user P from above via the instep abutting part 52 and the heel abutting part 53.

Accordingly, in the assist force application state, the force for generating the assist force is applied to the lumbar region and the buttocks of the user P wearing the lumbar orthosis 1 and the buttock orthosis 2 respectively and to the upper surface of the foot in abutting contact with the ankle orthosis 5. That is, no force is applied to a sole of the user P.

Therefore, in the assist device A1, by the force for generating the assist force, the COP of the user P is prevented from shifting. Thus, the user P easily maintains balance even in the assist force application state.

By the way, in the conventional assist device as described in Patent Document 1, the weight of a member constituting the assist device is configured to be released to a ground plane. Specifically, in the conventional assist device, the type that carries the foot is adopted as the foot orthosis, and the weight of a frame or the like connected to the foot orthosis is released to the ground plane via the foot orthosis.

In contrast, in the assist device A1, the lumbar orthosis 1 and the buttock orthosis 2 are respectively worn on the lumbar region and the buttocks of the user P, the ankle orthosis 5 is worn on the ankle of the user P, and the leg frame 7 connects the lumbar orthosis 1 and the buttock orthosis 2 with the ankle orthosis 5. That is, the weight of the leg frame 7 and the ankle orthosis 5 is supported by the lumbar region and the buttocks of the user P via the lumbar orthosis 1 and the buttock orthosis 2.

Hence, while the assist force is not being applied, the instep abutting part 52 and the heel abutting part 53 of the ankle orthosis 5 only slightly contact the upper surface of the foot of the user P, or they do not contact and are not in abutting contact to apply force.

Accordingly, in the assist device A1, there is no need to provide a member for grounding the assist device and release the weight of the member constituting the assist device to the ground plane. Thus, there is no need to adopt the type that carries the foot as the foot orthosis.

Therefore, according to the assist device A1, since there is no need to adopt the type that carries the foot as the foot orthosis, a structure located below the ankle is downsized, and a high degree of freedom is ensured relating to the motion of the ankle joint of the user P in the wearing state.

### Second Embodiment

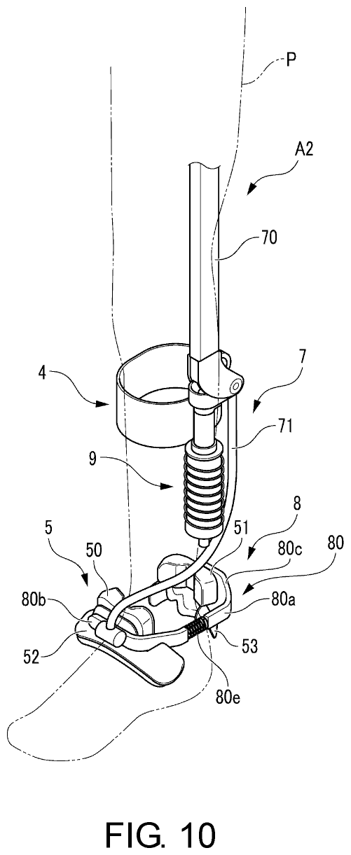

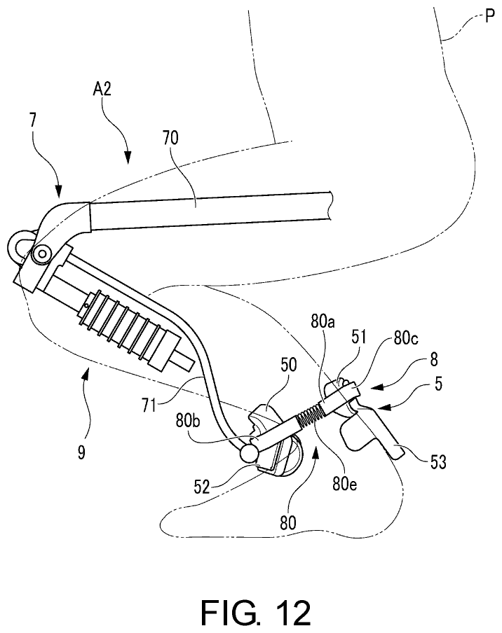

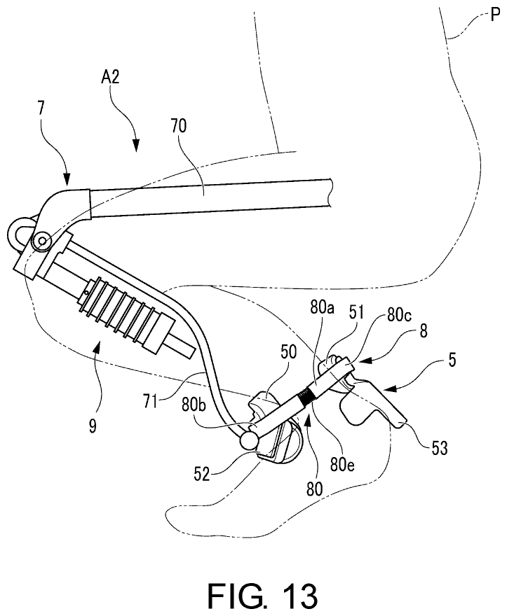

Hereinafter, the assist device A2 according to the second embodiment is described with reference to FIG. 10 to FIG. 13.

The assist device A2 of the present embodiment has the same configuration as the assist device A1 of the first embodiment, except that the configuration of the lower leg frame member of the leg frame and the configuration of the foot frame are different. Hence, in the following description, the same or corresponding configurations are denoted by the same reference numerals, and detailed description thereof is omitted.

As shown in FIG. 10, in the assist device A2, the lower leg frame member 71 of the leg frame 7 is curved forward and inward at a lower part. The lower leg frame member 71 is rotatably attached at its lower end to the front locking part 50 of the ankle orthosis 5 instead of the foot frame 8 about an axis extending along the pitch axis.

In addition, in the assist device A2, the foot frame 8 includes in the intermediate 80a a spring 80e expandable and contractible in the axial direction. The front part 80b of the foot frame 8 is rotatably attached to the front locking part 50, and the rear part 80c of the foot frame 8 is rotatably attached to the back locking part 51.

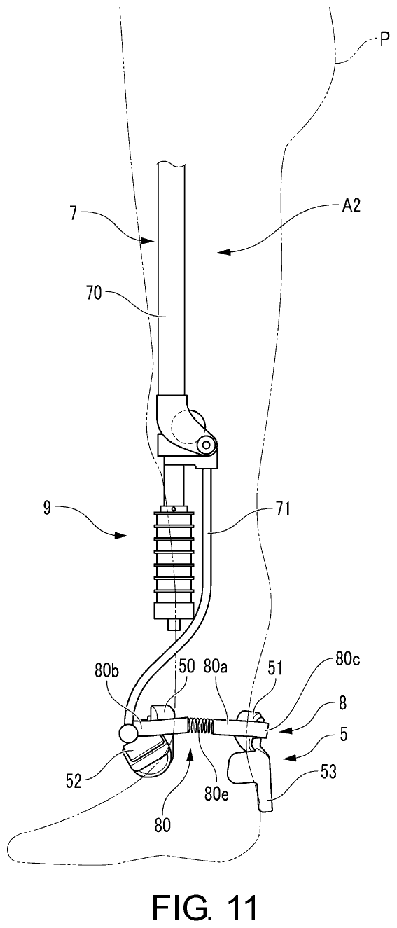

As shown in FIG. 11 to FIG. 13, the spring 80e expands and contracts, or bends, following a movement of the ankle of the user P in the wearing state.

By the assist device A2 configured in this way, the same effect as that of the assist device A1 of the first embodiment can be achieved. Like the assist device A1, in the assist device A2, the fourth connector 80d for connecting the leg frame 7 and the foot frame 8 can be omitted. Accordingly, the size, weight and cost can further be reduced.

### Third Embodiment

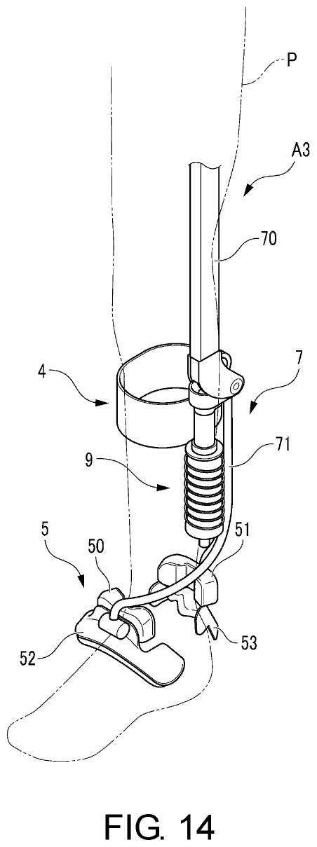

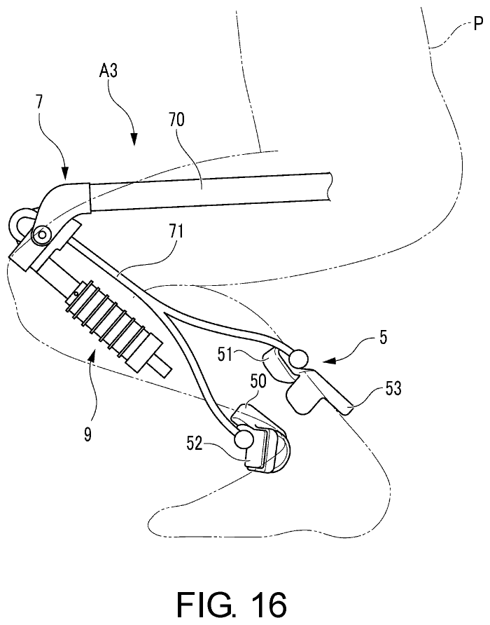

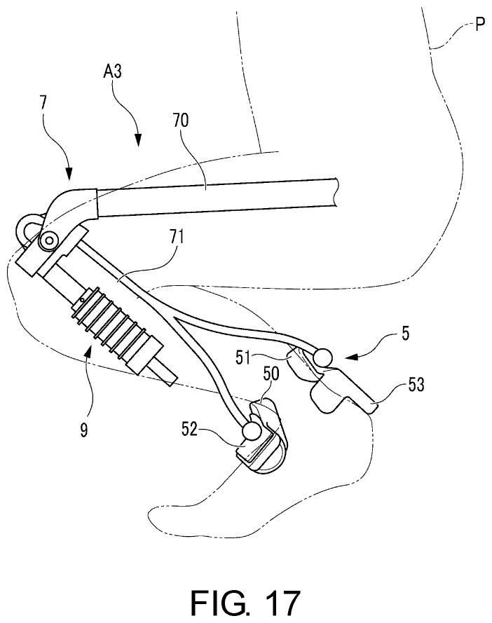

Hereinafter, an assist device A3 according to a third embodiment is described with reference to FIG. 14 to FIG. 17.

The assist device A3 of the present embodiment has the same configuration as the assist device A1 of the first embodiment, except that the configuration of the lower leg frame member of the leg frame is different and that the foot frame is omitted. Hence, in the following description, the same or corresponding configurations are denoted by the same reference numerals, and detailed description thereof is omitted.

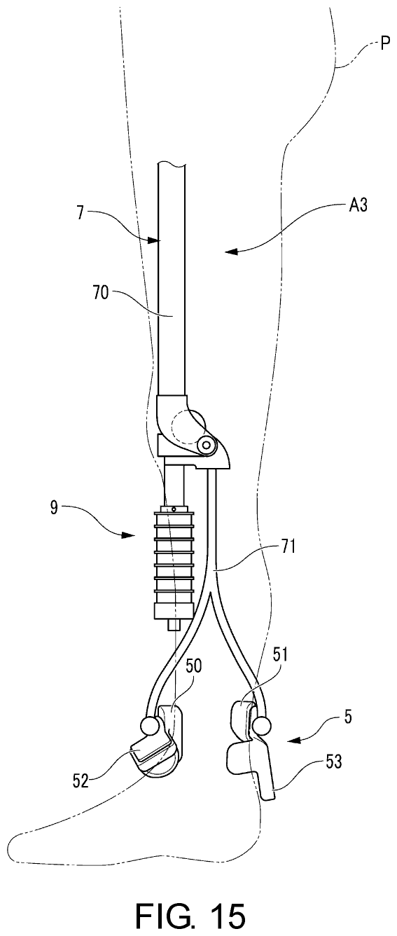

As shown in FIG. 14 and FIG. 15, in the assist device A3, the lower leg frame member 71 of the leg frame 7 is bifurcated in the front-rear direction at a portion lower than a base end and has an inverted Y shape.

In the bifurcated portion, a lower end of a front portion is curved forward and inward. At the lower end, the lower leg frame member 71 is rotatably attached to the front locking part 50 of the ankle orthosis 5 instead of the foot frame 8 about an axis extending in the pitch axis direction.

On the other hand, in the bifurcated portion, a lower end of a rear portion is curved rearward and inward. At the lower end, the lower leg frame member 71 is rotatably attached to the back locking part 51 of the ankle orthosis 5 instead of the foot frame 8 about an axis extending in the pitch axis direction.

Here, the lower leg frame member 71 of the assist device A3 is made of a material having somewhat lower rigidity and having elasticity as compared with the lower leg frame member 71 of the assist device A1.

Hence, as shown in FIG. 15 to FIG. 17, the lower leg frame member 71 bends following the movement of the ankle of the user P in the wearing state.

By the assist device A3 configured in this way, the same effect as that of the assist device A1 of the first embodiment can be achieved. Like the assist device A1, in the assist device A3, the foot frame 8 itself can be omitted. Accordingly, the size, weight and cost can further be reduced.

### Fourth Embodiment

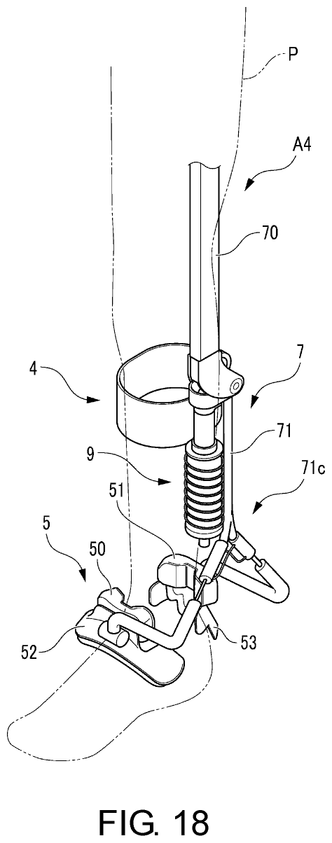

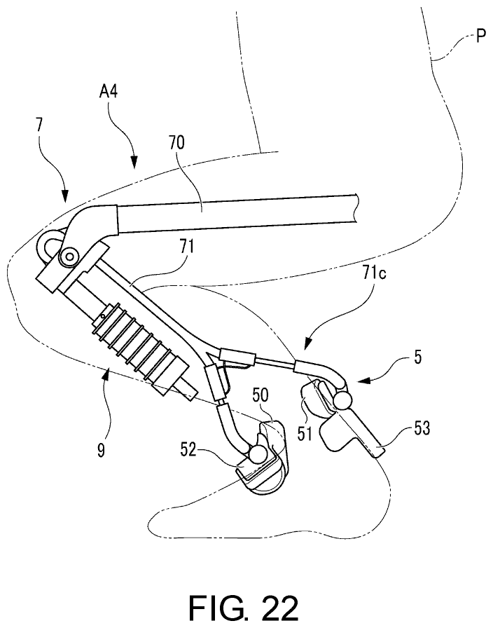

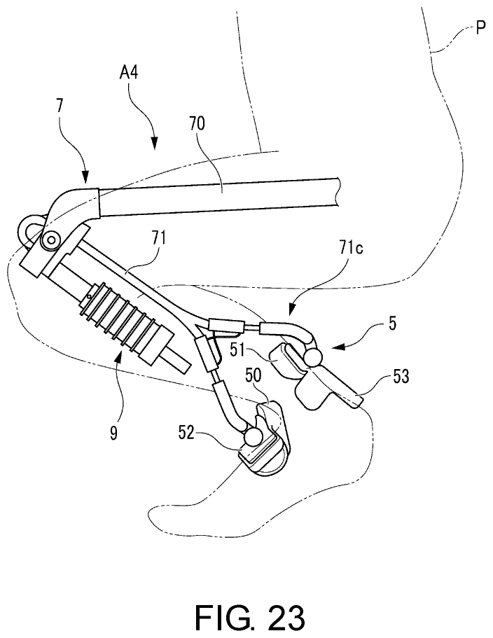

Hereinafter, an assist device A4 according to a fourth embodiment is described with reference to FIG. 18 to FIG. 23.

The assist device A4 of the present embodiment has the same configuration as the assist device A3 of the third embodiment, except that the configuration of the lower leg frame member of the leg frame is different. Hence, in the following description, the same or corresponding configurations are denoted by the same reference numerals, and detailed description thereof is omitted.

As shown in FIG. 18, in the assist device A4, the lower leg frame member 71 of the leg frame 7 is bifurcated in the front-rear direction at a portion lower than a base end and has an inverted Y shape. An expansion mechanism 71c is provided at the bifurcated portion.

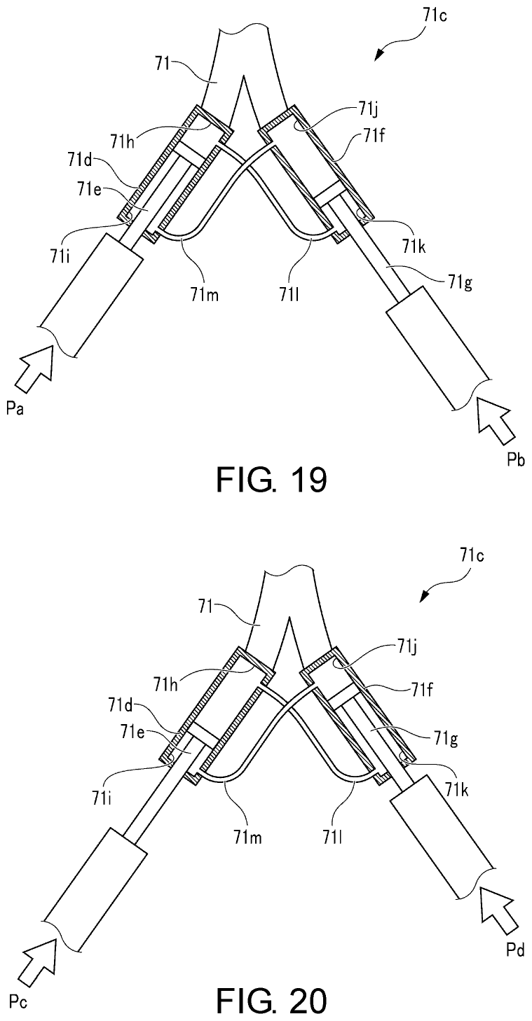

As shown in FIG. 19 and FIG. 20, the expansion mechanism 71c includes: a first cylinder 71d, provided on a front side of the lower leg frame member 71; a first piston 71e, capable of advancing and retreating in the axial direction inside the first cylinder 71d; a second cylinder 71f, provided on a rear side of the lower leg frame member 71; and a second piston 71g, capable of advancing and retreating in the axial direction inside the second cylinder 71f.

The inside of the first cylinder 71d is divided into two parts in the up-down direction by the first piston 71e. Accordingly, a first air cell 71h and a second air cell 71i located on an axial lower side of the first air cell 71h are defined inside the first cylinder 71d.

When one of the first air cell 71h and the second air cell 71i is expanded, the other may be compressed. Then, the first piston 71e advances or retreats according to the expansion and compression, and the front portion of the lower leg frame member 71 is expanded or contracted in the axial direction.

Similarly, the inside of the second cylinder 71f is divided into two parts in the up-down direction by the second piston 71g. Accordingly, a third air cell 71j and a fourth air cell 71k located on an axial lower side of the third air cell 71j are defined inside the second cylinder 71f.

When one of the third air cell 71j and the fourth air cell 71k is expanded, the other may be compressed. Then, according to the expansion and compression, the second piston 71g causes the rear portion of the lower leg frame member 71 to expand or contract in the axial direction.

Here, the first air cell 71h and the fourth air cell 71k are in communication with each other via a first pipe 711. Accordingly, the first air cell 71h and the fourth air cell 71k are configured so that the expansion and compression of one of them affects the other.

The second air cell 71i and the third air cell 71j are in communication with each other via a second pipe 71m. Accordingly, the second air cell 71i and the third air cell 71j are configured so that the expansion and compression of one of them affects the other.

Accordingly, the first air cell 71h, the second air cell 71i, the third air cell 71j and the fourth air cell 71k are configured so that the sum of a pressure Pa applied to the first piston 71e and a pressure Pb applied to the second piston 71g in a certain state (see FIG. 19) and the sum of a pressure Pc applied to the first piston 71e and a pressure Pd applied to the second piston 71g in a different state (see FIG. 20) are constant.

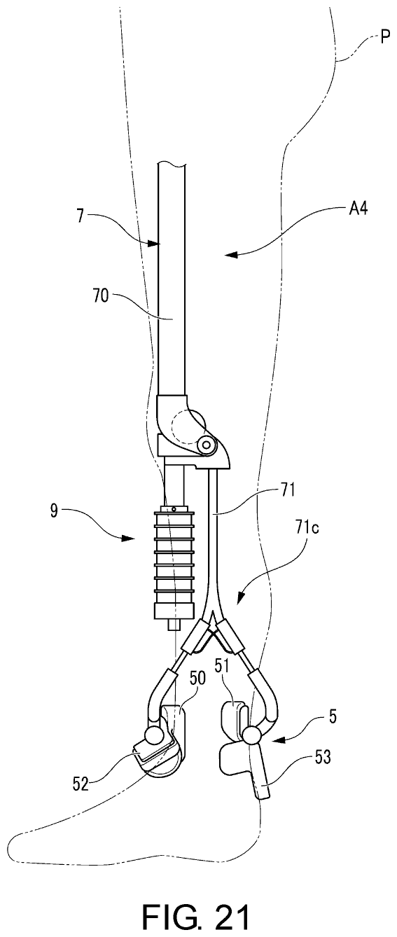

Hence, as shown in FIG. 21 to FIG. 23, the lower leg frame member 71 not only bends but also expands and contracts via the expansion mechanism 71c following the movement of the ankle of the user P in the wearing state.

By the assist device A4 configured in this way, the same effect as that of the assist device A1 of the first embodiment can be achieved. Like the assist device A1, in the assist device A4, the foot frame 8 itself can be omitted. Accordingly, the size can further be reduced.

### Other Embodiments

Although the above has described the embodiments illustrated in the drawings, the disclosure is not limited to such embodiments.

For example, in the above embodiments, as shown in FIG. 1 and the like, adopted is the assist device including a pair of left and right leg frames 7 located on a side of the user P in the wearing state. However, the assist device of the disclosure is not limited to such a configuration, and may be anything that applies the assist force assisting with the bending and stretching motions to the user's body.

Hence, for example, the assist device may include, as the first orthosis, a hip joint orthosis straddled by the user, in place of a lumbar orthosis and a buttock orthosis, and the assist device may be bendable or expandable and contractible between the legs of the user in the wearing state.

In the above embodiments, the lumbar orthosis 1 and the buttock orthosis 2 are adopted as the first orthosis. However, the first orthosis of the disclosure is not limited to such a configuration, and may be anything that is worn on the portion of the user's body above the knee joint.

Hence, for example, an orthosis worn on the abdomen, a vest type orthosis covering the upper body, or the like, may be adopted as the first orthosis in place of the lumbar orthosis. In addition, the lumbar orthosis 1 or the buttock orthosis 2 in the above embodiments may be omitted.

In the above embodiments, in order to easily fix the assist device to the body of the user P, the thigh orthosis 3 is provided. In order to operate the leg frame 7 integrally with the leg of the user P in the wearing state, the lower leg orthosis 4 is provided in addition to the ankle orthosis 5. However, the assist device of the disclosure is not limited to such a configuration, and may be anything that includes the first orthosis worn on the portion of the user's body above the knee joint and the second orthosis worn on the user's lower leg.

Hence, for example, in the above embodiments, at least one of the thigh orthosis 3 and the lower leg orthosis 4 may be omitted.

In the above embodiments, as the second orthosis, the ankle orthosis 5 worn on the ankle of the user P from the front and rear is adopted. However, the second orthosis of the disclosure is not limited to such a configuration, and may be anything that includes the locking part locked to the user's lower leg and the abutting part abutting against the user's foot from above in the assist force application state.

Hence, for example, the locking position of the second orthosis may be above the user's ankle instead of at the ankle Specifically, the second orthosis may have the following configuration. A locking part locked to the user's calf, and an abutting part abutting against a portion above the instep or heel and below the portion in abutting contact with the locking part are provided, and the locking part and the abutting part like the above are connected via a frame. In addition, the second orthosis may be worn either in front of or below the user's lower leg.

In the above embodiments, the ankle orthosis 5 includes, as the locking part, the front locking part 50 configured to be locked on the front surface side of the ankle of the user P in the wearing state, and the back locking part 51 configured to be locked on the back surface side of the ankle. In addition, the ankle orthosis 5 includes, as the abutting part, the instep abutting part 52 abutting against the instep of the user P in the assist force application state, and the heel abutting part 53 abutting against the portion on the back surface side of the foot of the user P that is inclined forward above the heel. However, it is not always necessary to provide two locking parts and two abutting parts according to the disclosure.

Hence, for example, there may be provided only one locking part or abutting part having a structure that can be wound around the ankle or the like, or there may be provided three or more locking parts or abutting parts abutting against the ankle or the like.

What is claimed is:

1. An assist device comprising:
a first orthosis worn on a portion of a body of a user above a knee joint;
a second orthosis worn on a lower leg of the user;
a leg frame connecting the first orthosis and the second orthosis; and
an assist mechanism, in response to bending and stretching motions of a leg of the user, applying an assist force that assists with the bending and stretching motions to the body of the user by operating the leg frame and separating the first orthosis and the second orthosis, wherein
the second orthosis comprises a locking part configured to be locked to the lower leg of the user, and an abutting part abutting against a foot of the user from above in a state in which the assist force is applied.

2. The assist device according to claim 1, comprising:
a foot frame extending laterally from the leg frame and rotatably connected to the leg frame about a pitch axis, wherein
the locking part comprises a front locking part configured to be locked to a front surface side of the lower leg of the user, and a back locking part configured to be locked to a back surface side of the lower leg of the user;
the second orthosis is connected to the leg frame via the foot frame;
the foot frame holds the front locking part on a front side and holds the back locking part on a rear side;
a connected portion between the leg frame and the foot frame is located between the front locking part and the back locking part in side view.

3. The assist device according to claim 1, comprising:
a foot frame extending laterally from the leg frame and rotatably connected to the leg frame about a pitch axis, wherein
the second orthosis is connected to the leg frame via the foot frame;
the locking part is configured to be locked to an ankle of the user;
a connected portion between the leg frame and the foot frame is located below the locking part.

4. The assist device according to claim 1, wherein
the locking part is configured to be locked to an ankle of the user;
the abutting part extends from a lower end of the locking part.

5. The assist device according to claim 4, wherein
the locking part is connected to the leg frame.

6. The assist device according to claim 4, wherein
the locking part comprises a front locking part configured to be locked to a front surface side of the lower leg of the user;
the abutting part comprises an instep abutting part abutting against an instep of the user.

7. The assist device according to claim 4, wherein
the locking part comprises a back locking part configured to be locked to a back surface side of the lower leg of the user;
the abutting part comprises a heel abutting part abutting against the foot or the ankle of the user from a heel side.

8. The assist device according to claim 1, wherein
the abutting part is located on a side of the leg frame and is configured to be non-rotatable about a roll axis.

9. The assist device according to claim 1, wherein
the abutting part comprises a collar extending from the abutting part so as to be located on a side surface of the foot of the user in a state in which the user wears the assist device.

10. The assist device according to claim 9, wherein
the abutting part comprises a heel abutting part abutting against the foot or an ankle of the user from a heel side;
the collar extends forward from the heel abutting part.

11. The assist device according to claim 1, wherein
the abutting part comprises an instep abutting part abutting against an instep of the user, wherein
the instep abutting part has a first curved shape convex upward in front view.

12. The assist device according to claim 11, wherein
the instep abutting part is located on a side of the leg frame;
the instep abutting part comprises a first portion located farther from the leg frame than an apex of the first curved shape in front view, and a second portion connected to the first portion and located closer to the leg frame than the apex of the first curved shape in front view, wherein
the first portion has a smaller radius of curvature than the second portion.

13. The assist device according to claim 12, wherein
the instep abutting part comprises a third portion connected to the second portion and located on a side opposite that of the first portion in front view, wherein
the third portion has a second curved shape convex downward in front view.

14. The assist device according to claim 1, wherein
the locking part comprises a pad configured to be locked to the lower leg of the user, wherein
the pad has a V shape convex toward a side opposite a side configured to be locked to the user in plan view.

15. The assist device according to claim 14, wherein
the locking part comprises a back locking part configured to be locked to a back surface side of the lower leg of the user, wherein
the back locking part is configured to be locked to an ankle of the user;
the pad is provided on a front surface side of the back locking part.

16. The assist device according to claim 14, wherein
the locking part comprises a front locking part configured to be locked to a front surface side of the lower leg of the user, and a back locking part configured to be locked to a back surface side of the lower leg of the user, wherein
the front locking part comprises, on a back surface side, a front pad configured to be locked to the front surface side of the lower leg of the user;
the back locking part comprises, on a front surface side, a back pad configured to be locked to the back surface side of the lower leg of the user;
at least one of the front pad and the back pad has the V shape convex toward the side opposite the side configured to be locked to the user in plan view.

17. The assist device according to claim 1, comprising:
a foot frame extending laterally from the leg frame and rotatably connected to the leg frame about a pitch axis, wherein
the second orthosis is connected to the leg frame via the foot frame;
the foot frame has a C shape in plan view.

18. The assist device according to claim 1, wherein
the locking part comprises a front locking part configured to be locked to a front surface side of the lower leg of the user, and a back locking part configured to be locked to a back surface side of the lower leg of the user, wherein
the front locking part is configured to be locked to an ankle of the user, and
an upper end of the front locking part is located below an upper end of the back locking part.
